(19) United States
(12) Patent Application Publication
(10) Pub. No.: US 2001/0002251 A1
(43) Pub. Date: May 31, 2001

(54) Intracellular sensitizers for sonodynamic therapy

(75) Inventors: Kathryn W. Woodburn, Sunnyvale, CA (US); David Kessel, Beverly Hills, MI (US)

Correspondence Address: AKIN GUMP STRAUSS HAUER & FELD, 1900 FROST BANK PLAZA, 816 CONGRESS AVENUE, AUSTIN, TX, 78701

(73) Assignee: Pharmacyclics, Inc.

(21) Appl. No.: 09/755,824

(22) Filed: Jan. 5, 2001

Related U.S. Application Data
(63) Continuation of application No. PCT/US99/15154, filed on Jul. 2, 1999.
(60) Provisional application No. 60/155,256, filed on Jul. 6, 1998.

(51) Int. Cl. A61K 9/14; A61K 49/00; A61B 8/13

(52) U.S. Cl. 424/9.5

(57) ABSTRACT

Texaphyrins are selectively retained in diseased tissue cells. When exposed to low level, non-thermal sonodynamic agent such as ultrasound, the texaphyrin-incorporated cells are selectively destroyed. There is provided a method of sonodynamic therapy by administering an effective amount of an intracellular sensitizer to a mammal in need thereof and providing an effective amount of a sonodynamic agent.

### FIELD OF THE INVENTION

The present invention relates generally to the use of a texaphrin in sonodynamic imaging and therapy, particularly as an intracellular sensitizer, and most particularly in the treatment of diseases characterized by neoplastic tissue, including but not limited to diseases related to the cardiovasculature, atheroma, stenosis, the prevention of intimal hyperplasia, restinosis, tumors, and activated macrophage mediated disorders such as rheumatoid arthritis.

### BACKGROUND INFORMATION

It has long been sought to selectively treat targeted tissue without adverse impact on surrounding healthy tissues. Progress towards this goal has been achieved using the class of agents known as sensitizers, but often at the risk of invasive administration of co-therapeutic agents.

Ultrasound has been used extensively over the last decades for medical diagnosis and physical therapy. Ultrasound has the ability to penetrate deeply into tissues while maintaining the ability to focus energy into small volumes. At high intensities, Ultrasound can be focused to penetrate deeply into tissues and cause cell cavitation or cell death. These thermal effects (i.e. cell killing) due to high intensity ultrasound absorption have been widely reported in conjunction with tumor treatment. However, thermogenesis of tumors by ultrasound has not been completely effective as a tumor treatment because the high intensity ultrasound energy causes cell cavitation in the tumors and in surrounding normal tissue, i.e., ultrasound is not selective for tumor cells.

Porphyrins are types of chemicals known to be somewhat selective for tumor tissue; they are reported to have been used in conjunction with low intensity ultrasound in an attempt to increase selectivity. The combination of sensitizers (porphyrins) with ultrasound to cause cell death has been termed “sonodynamic therapy”.

Further investigation into sonodynamic therapy using porphyrins showed that although the porphyrins have tumor selectivity, their use in conjunction with even low intensity ultrasound still gives rise to normal cell toxicity. It has been shown by fluorescent methodology that when the porphyrins are incorporated inside a cell, the application of low intensity ultrasound energy does not cause cell death. The low intensity ultrasound/porphyrin combination causes cell death only when the porphyrin is present in the extracellular matrix, which, under conditions of therapeutic administration is prior to clearance of the sensitizer from and results in toxicity to normal tissues.

Texaphyrins are aromatic pentadentate macrocyclic “expanded porphyrins” typically complexed with a metal (and sometimes referred to as “metallotexaphyrins”), which have been described as being useful as MRI contrast agents, as radiosensitizers, as chemosensitizers in oncology, and in photodynamic therapy. Texaphyrin is considered as being an aromatic benzannulene containing both 18π- and 22π-electron delocalization pathways. Texaphyrin molecules absorb strongly in the tissue-transparent 650-900 nm range, and they exhibit inherent selective uptake or biolocalization in certain tissues, particularly regions such as, for example, liver, atheroma or tumor tissue. Texaphyrins have exhibited significant tumor selectivity as detected by magnetic resonance imaging (for paramagnetic metal complexes) and by fluorescence. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each patent, publication, and application is incorporated herein by reference. Gadolinium texaphyrin has been shown to accumulate in the atheromas in human aortas by MRI (U.S. Pat. No. 5,252,720, previously incorporated by reference herein).

It remains desired to provide a non-invasive method of therapy using sensitizers that are selective to diseased tissue. This and other objects are satisfied in the present invention.

### SUMMARY OF THE INVENTION

Texaphyrins, administered in conjunction with low intensity ultrasound, cause cavitation leading to the selective death of cells in which the texaphyrin has been incorporated. Texaphyrins have a greater selectivity than porphyrins for diseased or neoplastic tissue, and have been shown to clear more quickly from normal tissue and extracellular matrix.

One aspect of the invention is a method of sonodynamic therapy by administering a texaphyrin to a mammal in need thereof in an amount effective for intracellular incorporation within selected target cells, and providing an amount of sonic energy sufficient to effect treatment without damage to normal, non-target cells.

The invention is also directed to methods for sonodynamically treating diseased tissue or neoplastic tissue cells, as well as other tissue cells or conditions that selectively intracellularly incorporate a texaphryin.

In another aspect, the invention relates to a method of sonodynamic therapy where the sonodynamic agent energy is delivered by a relatively simple to place, resilient, externally controllable, internal energy source, the internal energy source being ultrasound.

In still another aspect, the invention relates to a method of sonodynamic therapy by administering an effective amount of an intracellular sensitizer to a mammal in need thereof and providing an effective amount of a sonodynamic agent. In a preferred aspect, the method involves the additional step of waiting for said intracellular sensitizer to clear from the extracellular matrix and tissues surrounding the target cells to be treated.

In yet another aspect of the invention, the sonodynamic agent is ultrasound energy, and in a preferred aspect the ultrasound is administered at 3.9 W/cm².

In still another aspect of the invention the sonodynamic agent is administered in an amount effective in cells having incorporated the intracellular sensitizer, but ineffective in cells incorporating no intracellular sensitizer.

In a preferred aspect of the invention the intracellular sensitizer is a texaphyrin; presently preferred are Gadolinium Texaphyrin or Lutetium Texaphyrin.

Another aspect of the invention entails a method of selectively treating a mammal having a condition known to respond to texaphyrin sensitization therapy, by administering an effective amount of a texaphyrin followed by administering an effective amount of a sonodynamic agent to a physiologic site characteristic of said condition.

In still another aspect of the invention, the conditions treated are selected from the group neoplastic disease, cancer, cardiovascular disease, autoimmune disease, granulomatous disease, inflammatory disease, and transplant rejection.

Another aspect of the invention entails a method of imaging for the diagnosis of a condition in a mammal characterized by one or more accumulations of texaphyrin-absorbing cells, by administering a texaphyrin to the mammal in an amount effective for incorporation into such cells, waiting for the texaphyrin to clear from the extracellular matrix and tissues surrounding said target cells, and administering a sonodynamic agent in an amount sufficient to generate an image of the intracellular-texaphyrin-bearing cells, followed by generating a diagnostic image.

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

“Effective amount” means a dosage sufficient to provide treatment for the disease state being treated. This will vary depending on the patient, and the disease being effected.

“Intracellular sensitizer” means a therapeutic agent that is selectively incorporable into target cells, which increases or enhances the cytotoxicity of a sonodynamic agent to such target cells, e.g., a normally non-cytotoxic dosage can be cytotoxic to target cells in which the intracellular sensitizer has been incorporated.

“Sonodynamic agent” means ultrasound or any other externally controllable sonic energy source the toxicity of which is selectively enhanced by an intracellular sensitizer.

“Sonodynamic therapy” means the selective treatment of targeted tissue by administration of an intracellular sensitizer in combination with administration of a sonodynamic agent.

“Sensitizing,” “sensitized” and “sensitizes” mean the selective increase or enhancement of the cytotoxicity of a sonodynamic agent in target cells relative to surrounding non-target cells.

“Texaphyrin” means an aromatic pentadentate macrocyclic expanded porphyrins, also described as an aromatic benzannulene containing both 18π- and 22π-electron delocalization pathways. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each previously incorporated herein by reference.

“Treatment” or “treating” means any treatment of a disease in a mammal, including:

(i) preventing the disease, that is, causing the clinical symptoms of the disease not to develop;

(ii) inhibiting the disease, that is, arresting the development of clinical symptoms; and/or

(iii) relieving the disease, that is, causing the regression of clinical symptoms.

### Sonodynamic Therapy

In the present invention, sonodynamic therapy is provided to a mammal in need thereof by the co-administration of effective amounts of an intracellular sensitizer and a sonodynamic agent. It has surprisingly been discovered that unlike the prophyrins, which increase the toxicity of a sonodynamic agent only when present in the extracellular matrix (and not when incorporated intracellularly), texaphyrins are suitable intracellular sensitizers. Sonodynamic therapy offers certain advantages over existing radiation and photodynamic therapies. For example, ultrasound can penetrate tissues more effectively than light, facilitating greater access to non-invasive therapy, and can also be focused more effectively as compared to radiation. Diagnostic advantages are also achieved through the use of texaphyrins.

The precise mechanism of action of texaphyrin as an intracellular sensitizer for sonodynamic therapy remains to be definitively established. While not wanting to be bound by any particular theory, it is thought that the texaphyrin may induce cell cavitation, or that formation or prolongation of radical species may occur during sonication, facilitating cell death at sub-lethal sonodynamic agent dosages. Because texaphyrins are capable of sensitizing while intracellularly incorporated and are known to be cleared relatively rapidly from the plasma and extracellular matrix, particularly selective sensitization is achieved.

### lntracellular Sensitizers and Sonodynamic Agents

The texaphyrins employed in the present invention are as described above and in the disclosures incorporated by reference. Exemplary texaphyrins or texaphyrin metal complexes (or metallotexaphyrins) for use in the present invention are illustrated by Formulae I and II as follows:

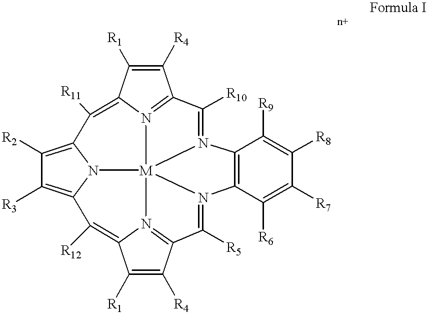

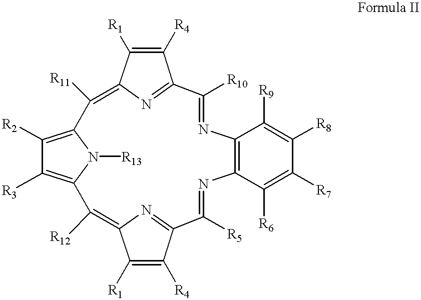

wherein M is H, a divalent metal cation, or a trivalent metal cation. Preferably, M is a divalent metal cation, or a trivalent metal cation. A preferred divalent metal cation is Ca(II), Mn(II), Co(II), Ni(II), Zn(II), Cd(II), Hg(II), Fe(II), Sm(II), or UO₂(II). A preferred trivalent metal cation is Mn(III), Co(III), Ni(III), Fe(III), Ho(III), Ce(III), Y(III), In(III), Pr(III), Nd(III), Sm(III), Eu(III), Gd(III), Tb(III), Dy(III), Er(III), Tm(III), Yb(III), Lu(III), La(III), or U(III). More preferred trivalent metal cations are Lu(III) or Gd(III). M or one of groups R₁ to R₁₃ can optionally have radioactive properties, and are as described in the U.S. patents, PCT publications, and allowed and pending patent applications previously incorporated by reference.

Preferred functionalizations are: when R₆ and R₉ are other than hydrogen, then R₅ and R₁₀ are hydrogen or methyl; and when R₅ and R₁₀ are other than hydrogen, then R₆ and R₉ are hydrogen, hydroxyl, or halide other than iodide. Other preferred functionalizations are where R₆ and R₉ are hydrogen, then R₅, R₁₀, R₁₁ and R₁₂ are independently hydrogen, phenyl, lower alkyl or lower hydroxyalkyl. The lower alkyl is preferably methyl or ethyl, more preferably methyl. The lower hydroxyalkyl is preferably of 1 to 6 carbons and 1 to 4 hydroxy groups, more preferably 3-hydroxypropyl. The phenyl may be substituted or unsubstituted.

More preferred are the compounds GdT2BET (where M=Gd(III)) and LuT2BET (where M=Lu(III)) (R₁ is CH₂(CH₂)₂OH, R₂ and R₃ are CH₂CH₃, R₄ is CH₃, R₇ and R₈ are O(CH₂CH₂O)₃CH₃, and R₅, R₆, and R₉-R₁₂ are H). Most preferred is compound GdT2BET (where M=Gd(III)). While the cited texaphyrins are presently preferred for use in the present invention, the invention is not limited thereto.

The preferred sonodynamic agents employed in the present invention is ultrasound, particularly is low intensity, non-thermal ultrasound, i.e., ultrasound generated within the wavelengths of about 0.1 MHz and 5.0 MHz and at intensities between about 3.0 and 5.0 W/cm². Ultrasound is generated by a focused array transducer, driven by a power amplifier. The diameter of the focused array transducer varies in size and spherical curvature to allow for variation of the focus of the ultrasonic output. Commercially available therapeutic ultrasound devices can be employed

### Utility, Testing and Administration

General Utility

Sonodynamic therapy employing intracellular sensitizers is effective in the treatment of conditions known to respond to texaphyrin sensitization therapy, including diseases characterized by neoplastic tissue, including the cancers sarcoma, lymphoma, leukemia, carcinoma and melanoma, cardiovascular diseases (e.g., arteriosclerosis, atherosclerosis, intimal hyperplasia and restenosis) and other activated macrophage-related disorders including autoimmune diseases (e.g., rheumatoid arthritis, Sjogrens, scleroderma, systemic lupus erythematosus, non-specific vasculitis, Kawasaki's disease, psoriasis, Type I diabetes, pemphigus vulgaris), granulomatous diseases (e.g., tuberculosis, sarcoidosis, lymphomatoid granulomatosis, Wegener's granulomatosus), inflammatory diseases (e.g., inflammatory lung diseases such as interstitial pneumonitis and asthma, inflammatory bowel disease such as Crohn's disease, and inflammatory arthritis), and in transplant rejection (e.g., in heart/lung transplants).

Testing

In vitro activity for sonodynamic therapy is determined, e.g., by measuring the effect of low-level, non-thermal ultrasound on the murine leukemia L1210 cell line in culture, measuring ultrasound-induced cytotoxicity, for example as described D. Kessel et al., International Journal of Radiation Biology, 66 (1994). After exposure to ultrasound, cells are placed in a CO₂ incubator at 37° C. for 3 days. Viability is assessed by an MTT assay in which the remaining cell population is treated with a tetrazolium dye (the dye is transformed by mitochondrial enzyme action to a blue product) for three hours. Color formation corresponds to the quantity of viable cells.

In vivo activity for sonodynamic therapy is determined, e.g., by post-mortum histological examination of tumor tissue by hematoxylin and eosin, as described in Yumita et al., Cancer Letters 112 (1997). The antitumor effects of sonodynamic therapy are evaluated by implanting tumor cells into one kidney in a mouse (the other kidney remaining untreatd and serving as a control). Approximately 24 hours after intravenous administration of a sonodynamic sensitizer to be tested, the mouse is anesthesized and the tumorous kidney exteriorized. The position and the angle of the mouse are adjusted to facilitate ultrasound ultrasound penetration of the entire kidney, with the tumor at the focal spot. Ultrasound is delivered in continuous waves by a focused array transducer, the kidney is returned to the abdominal cavity and the abdomen is closed. After 7 days the mouse is sacrificed and the kidneys stained with hematoxylin and eosin for histologic examination. The tumor bearing and control kidneys are compared.

Administration

The texaphyrin agents are administered at a therapeutically effective dosage, e.g., a dosage sufficient to provide treatment for the disease states previously described. The texaphyrin to be used in the method of the invention will be administered in a pharmaceutically effective amount, employing a method of administration, and means of activation by ultrasound as is known in the art.

The specific dose will vary depending on the particular texaphyrin chosen and the dosing regimen to be followed. Such dose can be determined without undue experimentation by methods known in the art or as described herein. Expected dose levels for an individual may range from about 0.01 mg/kg/treatment up to about 23 mg/kg/treatment or 0.05 μmol/kg to about 20 μmol/kg, depending on the texaphyrin chosen, administered in single or multiple doses (e.g. before each fraction of ultrasound).

For example, Lutetium Texaphyrin is administered in solution containing 2 mg/ml optionally in 5% mannitol, USP. Dosages of about 1.0 or 2.0 mg/kg to about 4.0 or 5.0 mg/kg, preferably 3.0 mg/kg may be employed, up to a maximum tolerated dose that was determined in one study to be 5.2 mg/kg. The texaphyrin is administered by intravenous injection, followed by a waiting period of from as little as several minutes or about 3 hours to as long as about 72 or 96 hours (depending on the treatment being effected) to facilitate intracellular uptake and clearance from the plasma and extracellular matrix prior to the administration of the sonodynamic agent. The co-administration of a sedative (e.g., benzodiazapenes) and narcotic analgesic are sometimes recommended prior to topical (as opposed to intravascular) light treatment. Topical administration. of Emla cream (lidocaine, 2.5% and prilocaine, 2.5%) under an occlusive dressing and other intradermal, subcutaneous and topical anesthetics may also be employed as necessary to reduce discomfort. Similar patient comfort considerations may apply in sonodynamic therapy, as will be apparent to the treating physician. Subsequent treatments can be provided after suitable time interval, currently approximately 21 days. In circumstances involving particular sensitivity to light, the treating physician may advise that certain patients avoid bright light for about one week following treatment.

Gadolinium Texaphyrin is administered in a solution containing 2 mM optionally in 5% mannitol, USP. Dosages of 0.1 mg/kg up to as high as 23.0 mg/kg have been delivered, preferably about 3.0 to about 15.0 mg/kg (for volume of about 90 to 450 mL) may be employed, optionally with pre-medication using anti-emetics above about 8.0 mg/kg. The texaphyrin is administered via intravenous injection over about a 5 to 10 minute period, followed by a waiting period of about 2 to 5 hours to facilitate intracellular uptake and clearance from the plasma and extracellular matrix prior to the administration of the sonodynamic agent.

Texaphyrins should be administered before administration of the sonodynamic agent. The texaphyrin may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer, which can vary in diameter and spherical curvature to allow for variation of the focus of the ultrasonic output. Commercially available therapeutic ultrasound devices may be employed in the practice of the invention. The duration and wave frequency, including the type of wave employed may vary, and the preferred duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave.

Texaphyrins are provided as pharmaceutical preparations. A pharmaceutical preparation of a texaphyrin may be administered alone or in combination with pharmaceutically acceptable carriers, in either single or multiple doses. Suitable pharmaceutical carriers include inert solid diluents or fillers, sterile aqueous solution and various organic solvents. The pharmaceutical compositions formed by combining a texaphyrin of the present invention and the pharmaceutically acceptable carriers (including infusion and perfusion fluids) are then easily administered in a variety of dosage forms such as injectable solutions.

For parenteral administration, solutions of the texaphyrin in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy use with a syringe exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, cyclodextrin derivatives, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars such as mannitol or dextrose or sodium chloride. A more preferable isotonic agent is a mannitol solution of about 2-8% concentration, and, most preferably, of about 5% concentration. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Texaphyrins may be impregnated into a stent by diffusion, for example, or coated onto the stent such as in a gel form, for example, using procedures known to one of skill in the art in light of the present disclosure.

### EXAMPLES

The following examples are given to enable those skilled in the art to more clearly understand and to practice the present invention. They should not be considered as limiting the scope of the invention, but merely as being illustrative and representative thereof.

### Example 1

### Determination of Sonodynamic Sensitizing Activity In Vitro Utilizing the Murine Leukemia L1210 Survival Assay

The effect of low-level, non-thermal ultrasound on the murine leukemia L1210 cell line in culture, measuring ultrasound-induced cytotoxicity is determined by a modification of the procedure initially described by Kessel et al., 1994. Int. J. Radiat. Biol., 66 (1994). Sonodynamic therapy with and without a texaphyrin and with and without low intensity irradiation of ultrasound. A murine 1210 cell line was used, The cytotoxicity of low-intensity ultrasound on murine leukemia L1210 cells is associated with cell fragmentation, and was detected by measuring the inhibition of the active transport of a non-metabolized amino acid (cytoleucine) and by a viability assay.

Cell Culture Systems—Murine leukemia L1210 cells were grown in Fischer's medium (GIBCO, Grand Island, N.Y.) supplemented with glutamine, 10% horse serum and gentamicin. All operations were carried out at 37° C. Exponentially growing cells were collected by centrifugation, resuspended (5×10⁶ cells/ml) in serum-free Fischer's medium buffered to pH 7.0 with 20 mM HEPES.

Sensitizers—Lutetium Texaphyrin (Lu-Tex or PCI-0123) and Gadolinium Texaphyrin (Gd-Tex or PCI-0120) were dissolved in water at a concentration of 6 μM.

Test Groups—Four groups of cells were established: Control A (no sensitizer and no ultrasound), Control B (no sensitizer, with ultrasound); 6 μM Lu-Tex with ultrasound, and 6 μM Gd-Tex with ultrasound. A small volume of the respective sensitizers were added to the Lu-Tex and Gd-Tex groups followed by a loading incubation of 15 minutes at 37° C. The cells were then suspended in fresh (serum free) medium, such that only intracellular texaphyrin remained.

Ultrasound—Exposure of cell suspensions (except Control A) to ultrasound was carried out in 2.5 cm diameter petri dishes in a bath of degassed water. A 2.54-cm-diameter transducer (Valpey-Fisher) was driven by a power amplifier (ENI 240L) with a continuous wave 1.94 MHz sinusoidal signal source (Wavetek Model 23). The acoustic field was calibrated with a Marconi PVDF membrane hydrophone in a free field condition 3.0 cm from the surface of the transducer which is also the distance from the face to the exposure dish. The acoustic intensity, expressed in terms of the spatial peak temporal average (SPTA), was 3.9 W/cm².

Fragmentation and Viability Studies—Cell counts and the particle size distribution were determined with a Coulter Electronics ZM Particle Analyzer and Model 256 Channelyzer. The Channelyzer determines the mean particle volume; intact cells were detected in channels 80-110. Smaler particles represent debris resulting from cell fragmentation. Viability testing involved dilution into fresh medium such that the initial density was <6,000/ml. Cell counts were again determined after incubation for sufficient time to allow the number of control (untreated) cells to reach 20,000/ml, approximately 5 doublings. The results are shown in Table 1. (It was established in parallel studies that use of a soft-agar assay system yielded viability data not significantly different from the growth curve information in this example.)

Amino Acid Transport—The effect of ultrasound on the capacity of L1210 cells for concentrative accumulation of the non-metabolized amino-acid cytoleucine was assesed as a probe for damage to membrane support systems. After exposure to ultrasound, cell suspensions in amino acid-free medium were warmed to 37° C. and radioactive CL (1 μM) was added. After 15 minutes, the cells were collected by centrifugation, washed with isotonic NaCl and cell radioactivity determined by liquid scintillation counting. Control (untreated) cells can concentrate CL by a factor of approximately 5; the effect of ultrasound on transport is reported in terms of % control accumulation. The results are shown in Table 1

TABLE 1
Intracellular Effect of Texaphyrin and Snondynamic Therapy
In Murine L1210 Cells

| | | Amount of | | |
|---|---|---|---|---|
| | Ultrasound | Cytoleucene | | |
| Group | Use | Transported | Fragmentation | Viability |
| Control A | no | 5.3 ± 0.4 | 0 | 100 |
| Control B | Yes | 4.9 ± 0.5 | 22 ± 4 | 76 ± 4 |
| Lu-Tex | Yes | 2.8 ± 0.3 | 73 ± 6 | 24 ± 3 |
| Gd-Tex | Yes | 3.2 ± 0.4 | 64 ± 4 | 42 ± 5 |

No detectable effect of ultrasound on the viability of L1210 cells was obtained until a power level of 3.7 W/cm was reached, although lower power levels impaired concentrative CL accumulation and altered partitioning results. A power level of 3.9 W/cm permitted a clear delineation of drug-enhanced cell damage. As shown in Table 1, at 3.9 W/cm, a substantial potentiation of the cytotoxic effect of ultrasound was observed when cells had previously been exposed to 6 μM levels of Lu-Tex or Gd-Tex. Loss of cell viability was associated with fragmentation and inhibition of cytoleucine transport. Unlike photodynamic therapy, the dose response curve for sonodynamic therapy with texaphyrins had no detectable shoulder, indicating an “all or none” effect.

From the foregoing it was concluded that ultrasound exposure to cells that have intracellularly taken up texaphyrin results in cytotoxicity. Unlike previously evaluated porphyrins, it is not required that texaphyrins be present in the extracellular medium in order to potentiate the cytotoxicity of ultrasound. Thus, texaphyrins can be administered, taken up by target cells, allowed to clear from plasma, extracellular matrix and non-target cells, and selectively potentiate the cytotoxic effects of ultrasound selectively in the target cells.

### Example 2

### Determination of Sonodynamic Sensitizing Activity Utilizing Experimental Kidney Tumor Assay

This procedure is a modification of a procedure initially described by Yumita et al., Cancer Letters 112 (1997).

Colon 26 tumor cells are implanted into one kidney in a male CDF mouse (the other kidney remaining untreated and serving as a control). After 14 days, 1.0 mg/kg of a texaphyrin is administered by intravenous injection. 24 Hours later, the mouse is anesthetized and the tumorous kidney exteriorized. The position and the angle of the mouse are adjusted to facilitate ultrasound ultrasound penetration of the entire kidney, with the tumor at the focal spot. Ultrasound is delivered in continuous waves by a focused array transducer. The tumor is exposed to focused ultrasound for 5 minutes in a progressive wave mode, at a frequency of 0.5 MHz for the first wave and 1 MHz for the second wave, at an intensity of 8 W/cm² followed by the second-harmonic superimposition of ultrasound at the same intensity, for a total exposure of 5 minutes. The kidney is returned to the abdominal cavity and the abdomen is closed. After 7 days the mouse is sacrificed and the kidneys are removed and stained with hematoxylin and eosin for histologic examination. The tumor-bearing and control kidneys are compared.

When texaphyrin (e.g., gadolinium texaphyrin, lutetium texaphyrin or yttrium texaphyrin) is tested in accordance with the foregoing procedure, the tumor-bearing and control kidneys are substantially the same, indicating successful sonodynamic treatment of the experimentally induced tumor.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

What is claimed is:

1. A method of sonodynamic therapy comprising administering an effective amount of an intracellular sensitizer to a mammal in need thereof and providing an effective amount of a sonodynamic agent.

2. The method of claim 1 comprising the additional step of waiting for said intracellular sensitizer to clear from the extracellular matrix and tissues surrounding the target cells to be treated.

3. The method of claim 2 wherein the target cells to be treated are cells of a cancer, a cardiovascular disease, an autoimmune disease, a granulomatous disease, an inflammatory disease, or transplant.

4. The method of claim 1 wherein the sonodynamic agent is ultrasound energy.

5. The method of claim 4 wherein the ultrasound is administered at 3.9 W/cm².

6. The method of claim 1 wherein the sonodynamic agent is administered in an amount effective in cells having incorporated the intracellular sensitizer, but ineffective in cells incorporating no intracellular sensitizer.

7. The method of claim 1 wherein the intracellular sensitizer is a texaphyrin.

8. The method of claim 6 wherein the texaphyrin is Gadolinium Texaphyrin or Lutetium Texaphyrin.

9. A method of selectively treating a mammal having a condition known to respond to texaphyrin sensitization therapy, said method comprising administering an effective amount of a texaphyrin followed by administering an effective amount of a sonodynamic agent to a physiologic site characteristic of said condition.

10. The method of claim 9 comprising the additional step of waiting for said texaphyrin to clear from the extracellular matrix and tissues surrounding the target cells to be treated at said physiologic site.

11. The method of claim 9 wherein the condition is selected from the group neoplastic disease, cancer, cardiovascular disease, autoimmune disease, granulomatous disease, inflammatory disease, and transplant rejection.

12. The method of claim 9 wherein the sonodynamic agent is ultrasound energy.

13. The method of claim 9 wherein the ultrasound is administered at 3.9 W/cm².

14. The method of claim 9 wherein the sonodynamic agent is administered in an amount effective in cells having incorporated the texaphyrin, but ineffective in cells incorporating no texaphyrin.

15. The method of claim 9 wherein the texaphyrin is Gadolinium Texaphyrin or Lutetium Texaphyrin.

16. A method of imaging for the diagnosis of a condition in a mammal characterized by one or more accumulations of texaphyrin-absorbing cells, comprising administering a texaphyrin to the mammal in an amount effective for incorporation into such cells, waiting for said texaphyrin to clear from the extracellular matrix and tissues surrounding said target cells, and administering a sonodynamic agent in an amount sufficient to generate an image of the intracellular-texaphyrin-bearing cells, and generating a diagnostic image.

17. The method of claim 16 further comprising the step of administrating an amount of a sonodynamic agent effective to treat the condition diagnosed.
